(19) United States
(12) Patent Application Publication
(10) Pub. No.: US 2021/0346260 A1
(43) Pub. Date: Nov. 11, 2021

(54) Oral Care Compositions Comprising Dicarboxylic Acid

(71) Applicant: The Procter & Gamble Company, Cincinnati, OH (US)

(72) Inventors: Michael David Curtis, Mason, OH (US); Andrew Frederic Groth, Mason, OH (US); Paul Albert Sagel, Maineville, OH (US); Samuel James St. John, Cincinnati, OH (US)

(21) Appl. No.: 17/308,079

(22) Filed: May 5, 2021

Related U.S. Application Data
(60) Provisional application No. 63/020,034, filed on May 5, 2020.

(51) Int. Cl. A61K 8/362; A61K 8/21; A61Q 11/00; A61K 8/19; A61K 8/20; A61K 8/24; A61K 8/27; A61K 8/25; A61K 8/49; A61K 8/44; A61K 8/22

(52) U.S. Cl. CPC A61K 8/362; A61K 2800/28; A61Q 11/00; A61K 8/19; A61K 8/20; A61K 8/24; A61K 8/27; A61K 8/25; A61K 8/492; A61K 8/4946; A61K 8/445; A61K 8/22; A61K 8/44; A61K 2800/30; A61K 2800/48; A61K 8/21

(57) ABSTRACT

Oral care compositions including dicarboxylic acid and fluoride. Oral care kits including a first oral care composition with fluoride and a second oral care composition with dicarboxylic acid. Oral care compositions including fluoride and dicarboxylic acid that provide an enhanced anticaries benefit, a stain prevention benefit, and/or a stain removal benefit.

### FIELD OF THE INVENTION

The present invention relates to oral care compositions comprising dicarboxylic acid and fluoride. The present invention also relates to oral care compositions with an unexpected improvement in anticaries activity and/or fluoride uptake.

### BACKGROUND OF THE INVENTION

Oral care compositions can include fluoride as an anticaries agent. Specifically, fluoride ions sources, such as sodium fluoride, stannous fluoride, and/or sodium monofluorophosphate, among others, can be added to dentifrice compositions to deliver anticaries benefits.

Fluoride ions provide an anticaries benefit through the uptake of fluoride ions into enamel. The interaction of fluoride with the mineral component of teeth (known as hydroxyapatite or HAP) produces a fluorohydroxyapatite (FAP) mineral, through the substitution of OH⁻ in HAP with F⁻. Fluoride incorporation into the dental enamel as FAP results in increased hydrogen bonding, a denser crystal lattice, and an overall decrease in the solubility of dental enamel. The incorporation of fluoride into the hydroxyapatite (HAP) lattice may occur while the tooth is forming or by ion exchange after it has erupted. Thus, fluoride is routinely added to dentifrice and mouth rinses to strengthen dental enamel.

The ability to add fluoride to oral care compositions is limited by regulation. In many countries, oral care compositions can only have a defined amount and/or concentration of fluoride ions because fluoride is regulated as an anticaries drug. Compositions with higher concentrations of fluoride ions can provide higher anticaries effect but can have safety concerns if unintentionally swallowed. Thus, these compositions must be prescribed and/or applied by a dental professional. As such, there is a need a for an oral care composition which include the regulated amount of fluoride, but with an increased anticavity activity with ingredients designed to enhance or complement fluoride's activity or efficacy.

### SUMMARY OF THE INVENTION

Disclosed herein is an oral care composition comprising (a) dicarboxylic acid; and (b) fluoride, wherein the pH of the oral care composition is from about 4 to about 7.

Also disclosed herein is an oral care kit comprising (a) a first oral care composition comprising fluoride; and (b) a second oral care composition comprising dicarboxylic acid.

Also disclosed herein is an oral care regimen comprising (a) applying a first oral care composition, the first composition comprising fluoride, to an oral cavity of a user; and (b) applying a second oral care composition, the second composition comprising dicarboxylic acid to the oral cavity of the user.

### DETAILED DESCRIPTION OF THE INVENTION

The present invention is directed to oral care compositions that have fluoride and provide an unexpectedly high anti-cavity benefit relative to the amount of fluoride present. Dental caries, or tooth decay, is a breakdown of the teeth due to the acids made by bacteria. Cavities are caused by the acid produced by bacteria dissolving the hard tissues of the teeth, such as enamel, dentin, and/or cementum. The acid is produced by the bacteria when the bacteria breaks down food debris or sugar on the tooth's surface.

Fluoride ions provide an anticaries benefit by making the tooth's surface less soluble to the acid produced by the bacteria, “plaque acid”. Tooth's enamel is made from hydroxyapatite (Ca₅(PO₄)₃(OH)). Hydroxyapatite can be dissolved from the enamel at a pH of under 5.5 (demineralization). If hydroxyapatite is demineralized in the presence of fluoride ions, fluorapatite (Ca₅(PO₄)₃(F)) can remineralize on the surface of a tooth's enamel. In sum, this process is a replacement of a hydroxyl (OH) ion with a fluoride (F) ion. Fluorapatite is inherently less soluble than hydroxyapatite, even under acidic conditions. Thus, fluoride works as an anticaries drug to make the tooth's surface more resistant and less soluble to plaque acid.

Oxalic acid and salts thereof have been disclosed in the literature, such as in U.S. Pat. No. 5,026,539, as anti-caries agents through its ability to reduce the acidogenicity of plaque biofilms. The mechanism of this is not well understood. Additionally, Poile et al, EP 0242977, disclose anticaries compositions with fluoride in the pH range of 4 to 10. The reason for the pH limitation is not disclosed.

Importantly, as disclosed herein, dentifrice compositions comprising fluoride and dicarboxylic acid, such as oxalate, at a pH of less than 4.5 led to the demineralization of enamel surfaces. Thus, combinations of oxalate and fluoride did not work over the entire pH range disclosed by Poile. This invention, therefore, provides a composition including dicarboxylic acid, such as oxalic acid and its salts, in combination with fluoride, that resulted in increased anticaries benefit, but did not lead to damage to enamel. Unexpectedly, it was necessary to restrict the pH range to prevent enamel demineralization (below a pH of 4.5).

While not wishing to be bound by theory, it is believed that the disclosed oral care compositions have an additional mechanism for lowering the solubility of the enamel and contributing to the reduction in enamel loss during plaque acid attack. It is believed that the oxalate anions of the disclosed oral care compositions stabilize the enamel surface when applied at a moderate pH range to subsequent acid damage. The oxalate anions, reacting with calcium from the tooth, can form an acid-insoluble, stable phase that forms at low pH according to Formula 1.

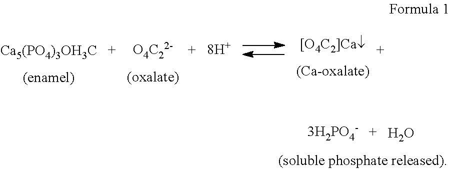

The anti-solubility effect at ow concentration is unique to the oxalate anion as calcium salts of the other forms of mono-, di-, and tri-carboxylic acids are significantly more soluble at low pH and do not form an insoluble precipitate on the tooth surface. Unexpectedly, an enhancement of the resistance to plaque acid attack by oxalate-containing oral care compositions was observed in laboratory models, as described herein.

While not wishing to be being bound by theory, it is believed that at certain pH conditions oxalate anions extract calcium ion from the enamel mineral in order to form this insoluble phase. Until the insoluble phase is formed, the oxalate enhances the surface solubility of the enamel surface by reducing the local degree of saturation of enamel with respect to calcium. At certain pH conditions and low calcium content (e.g., during exposure to an oral care composition with a pH of below about 4.5), the application of the oxalate anion may result in too much calcium loss, which can result in measurable softening of the enamel surface. We unexpectedly discovered during the application of the low pH, oxalate-containing oral care composition to generate the acid-insoluble layer resulted in measurable surface demineralization not previously disclosed in the art. Consequently, the anticaries benefit of oxalate and fluoride occurs only at a pH of about 4.5 to about 7 where demineralization of the enamel surface is balanced with remineralization. At a pH of below about 4.5, oxalate provides too much demineralization, which limits any anticaries benefit provided by the more available fluoride ions.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen’ or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise dicarboxylic acid, tin, and/or fluoride. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

### Dicarboxylic Acid

The oral care composition comprises dicarboxylic acid. The dicarboxylic acid comprises a compound with two carboxylic acid functional groups. The dicarboxylic acid can comprise a compound or salt thereof defined by Formula I.

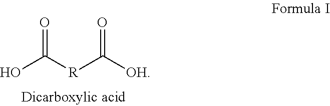

R can be null, alkyl, alkenyl, allyl, phenyl, benzyl, aliphatic, aromatic, polyethylene glycol, polymer, 0, N, P, or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, bras sylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, fumaric acid, terephthalic acid, glutaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.01% to about 10%, from about 0.1% to about 15%, from about 1% to about 5%, or from about 0.0001 to about 25%, of dicarboxylic acid.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

pH

The pH of the oral care compositions as described herein can be from about 4 to about 7, from about 4 to about 6, from about 4.5 to about 6.5, or from about 4.5 to about 5.5. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

The pH of the oral care compositions as described herein have a preferred pH of below about 7 or below about 6 due to the pKa of the dicarboxylic acid. While not wishing to be bound by theory, it is believed that the dicarboxylic acid displays unique behavior when the pH is below about 7 or below about 6, but surfaces in the oral cavity can only also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential to damage teeth by acid dissolution is greatly increased. Consequently, the oral care compositions comprising dicarboxylic acid, as described herein, preferably have a pH from about 4 to about 7, from about 4 to about 6, from about 4.5 to about 6.5, or from about 4.5 to about 5.5 to minimize metal hydroxide/metal oxide formation and any damage to oral hard tissues (enamel, dentin, and cementum).

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Monodentate Ligand

The oral care composition can comprise monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R-COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 0.01% to about 10%, from about 0.1% to about 15%, from about 1% to about 5%, or from about 0.0001 to about 25%, by weight of the composition, of the monodentate ligand.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e. with two functional groups), tridentate (i.e. with three functional groups), tetradentate (i.e. with four functional groups), etc. Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC-R-COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, citric acid, phytic acid, pyrophosphate, tripolyphosphate, tetrapolyphosphate, hexametaphoshate, salts thereof, and/or combinations thereof.

The oral care composition can include from about 0.01% to about 10%, from about 0.1% to about 15%, from about 1% to about 5%, or from about 0.0001 to about 25%, by weight of the composition, of the polydentate ligand.

### Ratio of Tin to Monodentate Ligand to Polydentate Ligand

The oral care composition, as described herein, can comprise a ratio of tin to monodentate ligand to polydentate ligand that provides an unexpectedly high amount of soluble tin and/or a superior fluoride uptake. Suitable ratios of tin to monodentate ligand to polydentate ligand can be from about 1:0.5:0.5 to about 1:5:5, from about 1:0.5:0.75 to about 1:5:5, from about 1:1:1 to about 1:5:5, from about 1:1:0.5 to about 1:2.5:2.5, from about 1:1:1 to about 1:2:2, from about 1:0.5:0.5 to about 1:3:1, or from about 1:0.5:0.5 to about 1:1:3.

Desired herein are oral care compositions with a soluble Sn of at least about 1000 ppm, 2000 ppm, 4000 ppm, at least about 4500 ppm, at least about 5000 ppm, at least about 6000 ppm, and/or at least about 8000 ppm. Also desired herein are oral care compositions with a fluoride uptake of at least about 6.5 μg/cm², at least about 7.0 μg/cm², at least about 8.0 μg/cm², or at least about 9.0 μg/cm² after a time period of at least about 9 days, 30 days, 65 days, 75 days, 100 days, 200 days, 365 days and/or 400 days.

In total, while not wishing to be bound by theory it is believed that the soluble Sn amount is correlated to bioavailable Sn as it is freely available to provide an oral health benefit. Fully bound Sn (i.e. Sn that is overchelated) or precipitated Sn (i.e. insoluble tin salts, such as Sn(OH)₂ and/or Sn-based stains can form when Sn is underchelated) would not be included in the measurement for soluble Sn. Additionally, while not wishing to be bound by theory, it is believed that a carefully balanced ratio of Sn to monodentate and polydentate ligands can provide a high amount of bioavailable fluoride and Sn ions without some of the negatives to the use of cationic antimicrobial agents, such as surface staining. Thus, additional screening experiments were done to quantify and qualify the ranges and identities of monodentate and polydentate ligands.

### Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula II, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

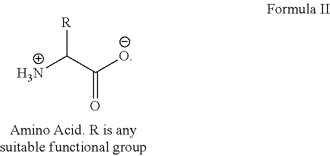

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acid which has an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the dicarboxylic acid include emulsion compositions, such as the emulsions compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Oral Care Regimen

The dicarboxylic acid can be delivered in the same composition as the tin and/or fluoride or the dicarboxylic acid can be delivered in a separate composition. For example, a first composition can comprise tin and/or fluoride and a second composition can comprise dicarboxylic acid. The first and second composition can be delivered simultaneously, such as in a dual-phase composition or sequentially from discrete compositions.

An oral care kit can include the first composition comprising tin and/or fluoride and the second composition comprising dicarboxylic acid. The oral care kit can also include instructions directing a user to apply the first composition to an oral cavity of the user followed by applying the second composition to the oral cavity of the user. The first composition can be expectorated prior to the application of the second composition or the second composition can be applied prior to the expectoration of the first composition from the oral cavity.

The entire oral care regimen can have a duration of from one minute to about three minutes with each application step having a duration of from about 30 seconds to about 2 minutes or about 1 minute.

The components can be delivered to the oral cavity simultaneously or sequentially. The simplest case is simultaneous, continuous delivery of equal amounts of the two components or a constant ratio of the components during a single oral care session. The two components may be provided separately, such as in a dual-phase composition in two separate compositions, and then delivered simultaneously to the oral cavity. Brushing duration is sufficiently short so that the components will not be inactivated. Another use for simultaneous, continuous delivery is systems that include two components that react relatively slowly, and that will remain in the oral cavity after brushing to be absorbed by the teeth and or gums.

In the case of sequential delivery, both components may be delivered during a single oral care session, e.g., a single brushing session or other single treatment session (single use, start to finish, by a particular user, typically about 0.1 to 5 minutes), or alternatively the components may be delivered individually over multiple oral care sessions. Many combinations are possible, for example delivery of both components during a first oral care session and delivery of only one of the components during a second oral care session.

Sequential delivery during a single oral care session may take various forms. In one case, two components are delivered in alternation, as either a few relatively long duration cycles during brushing (A B A B), or many rapid-fire alternations (A B A B A B A B A B . . . A B).

In another case, two or more components are delivered one after the other during a single oral care session, with no subsequent alternating delivery in that oral care session (A followed by B). For example, a first composition comprising fluoride and/or tin can be delivered initially, to initiate brushing and provide cleansing, followed by a second composition comprising dicarboxylic acid.

### Examples

The invention is further illustrated by the following examples, which are not to be construed inn any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Compositions

TABLE 1A
Oral Care Compositions

| Ingredient (wt %) | Ex. 1 | Ex. 2 | Ex. 3 | Ex. 4 |
|---|---|---|---|---|
| Sorbitol | 45.0000 | 47.0000 | 48.0000 | 48.0000 |
| Treated Water | 19.1091 | 20.2620 | 19.6550 | 21.1311 |
| SnF₂ | 0.4540 | — | — | 0.4540 |
| SnCl₂ | 0.5619 | — | — | 0.5619 |
| (10% silica blend) | | | | |
| NaF | — | 0.2430 | — | — |
| Sodium Gluconate | 1.3000 | — | — | 1.3000 |
| NaOH 50% | 0.1500 | — | — | 0.8700 |
| Saccharin | 0.3500 | 0.3500 | 0.3500 | 0.4000 |
| Sucralose | 0.0800 | 0.0800 | 0.0800 | 0.2000 |
| Xanthan Gum | 0.8750 | 0.8750 | 0.8750 | 0.8750 |
| Carrageenan | 1.5000 | 1.5000 | 1.5000 | 1.5000 |
| Citric Acid | — | 0.2750 | 0.1250 | — |
| Zinc Citrate | — | — | — | 0.5330 |
| Na Citrate | 1.2050 | — | — | — |
| Potassium oxalate | 3.1400 | 3.1400 | 3.1400 | — |
| monohydrate | | | | |
| TiO₂ | 0.5000 | 0.5000 | 0.5000 | 0.5000 |
| Silica | 17.5000 | 17.5000 | 17.5000 | 17.5000 |
| Sodium | 7.0000 | 7.0000 | 7.0000 | 5.0000 |
| Lauryl Sulfate | | | | |
| (28 wt % solution) | | | | |
| Flavor | 1.2750 | 1.2750 | 1.2750 | 1.1750 |

TABLE 1B
Oral Care Compositions

| Ingredient (wt %) | Ex. 5 |
|---|---|
| Flavor | 1.20% |
| Sodium Monofluorophosphate | 1.15% |
| Sorbitol Solution (70%) | 49.90% |
| Mica-Titanium Dioxide coated | 0.50% |
| Cocamidopropyl Betaine Solution (30%) | 1.50% |
| Potassium Oxalate | 3.00% |
| Silica Thickening | 1.50% |
| Silica Abrasive | 12.00% |
| Sodium lauryl sulfate solution (28%) | 5.50% |
| Sodium saccharin | 0.40% |
| Sucralose powder | 0.08% |
| Phosphoric Acid | 0.55% |
| Xanthan Gum | 0.75% |
| Carrageenan Iota | 1.50% |
| Water | 20.50% |

TABLE 2
Summary of Tested Oral Care Compositions

| | Summary of | Slurry pH of |
|---|---|---|
| Examples | Ingredients | Composition |
| Ex. 1 | SnF₂, SnCl₂, Oxalate | 6.52 |
| Ex. 2 | NaF, Oxalate | 6.94 |
| Ex. 3 | Oxalate | 7.23 |
| Ex. 4 | SnF₂, SnCl₂ | 6.76 |
| Ex. 5 | MFP, Oxalate | 4.40 |
| Crest ® Cavity | NaF | 7.00 |
| Protection (CCP) | | |

The treatment compositions included those from TABLE 1A and the summary TABLE 2. Ex. 1 included stannous fluoride, stannous chloride, and potassium oxalate (a dicarboxylic acid). Ex. 2 was similar to Ex. 1 except Ex. 2 replaced stannous fluoride/stannous chloride with sodium fluoride. Ex. 3 removes sodium fluoride from Ex. 2. Ex. 4 is the same as Ex. 1, but without potassium oxalate. Ex. 1-4 were compared to CCP (1100 ppm theoretical F).

The enamel softening treatment compositions included those from TABLE 1B and the summary TABLE 2. Ex. 1-5 were compared to water (negative) and citric acid (positive) softening controls.

### pH Cycling

This pH cycling method has successfully been used to demonstrate the anticaries potential of numerous fluoride-containing dentifrice formulations.

Prepared human enamel rod specimens (dentin rod specimens for dentin pH cycling) were subjected to a pH cycling regimen for 5 days consisting of dentifrice treatments, a period of demineralization and a period of remineralization. At the end of each demineralization period the demineralization solution was analyzed by ICP for calcium content. The cumulative amount of Ca lost from each specimen into the demineralization solution over 5 days cycling was a measure of the demineralization protection potential of the treatment.

The technique is sensitive to hydroxyapatite crystal growth inhibitors like pyrophosphates, polyphosphates, stannous, and zinc. All of which improve the resistance of the tooth to demineralization and consequently improve the performance of the dentifrice. This method does not assess the anticaries potential of ingredients that reduce plaque acidogenicity or acidity.

### Solutions Used in pH Cycling

TABLE 4
Fluoride presoak solution

| | | Molecular | | Target |
|---|---|---|---|---|
| Raw Materials | Formula | Weight | Molarity | (1 Liter) |
| Calcium Phosphate, Dibasic, | CaHPO₄ | 136.06 | 0.001 | 0.1361 g |
| Anhydrous | | | | |
| Sodium Fluoride, Anhydrous | NaF | 41.99 | 0.001 | 0.0420 g |
| Sodium Chloride, Anhydrous | NaCl | 58.44 | 0.046 | 2.6882 g |
| Hydrochloric Acid, 1.0N | HCl | 36.46 | — | — |
| Deionized Water | H₂O | — | — | — |

The demineralization solution served as an acid challenge similar to that generated by plaque acids. The addition of Carbopol helped protect the ground and polished enamel cores from losing too much mineral in the body of the lesion. Calcium and Phosphorus levels were theoretically equal 80 ppm Ca and 62 ppm P.

TABLE 5
Demineralization Solution

| | | Molecular | | Target | |
|---|---|---|---|---|---|
| Raw Materials | Formula | Weight | Molarity | (1 Liter) | |
| Glacial Acetic Acid | CH₃COOH | 60.05 | 0.075 | 4.31 | ml |
| Sodium Phosphate | NaP | 137.99 | 0.002 | 10.00 | ml |
| Stock Solution | | | | | |
| Sodium Hydroxide, | NaOH | 40.00 | — | 1.00 | ml |
| 50% (Step 4) | | | | | |
| Calcium Chloride | CaCl | 147.02 | 0.002 | 10.00 | ml |
| Stock Solution | | | | | |
| Carbopol 907 | C₉H₉NO₂ | Polymer | 0.20 | 2.00 | g |
| Sodium Hydroxide, | NaOH | 40.00 | — | 0.25 | ml |
| 50% (Step 9) | | | | | |
| Deionized Water | H₂O | — | — | 1000 | ml |

TABLE 6A
Sodium Phosphate Stock Solution (200 mM)

| | | Molecular | Target | Target | |
|---|---|---|---|---|---|
| Raw Materials | Formula | Weight | Molarity | (500 ml) | |
| Sodium Phosphate, | NaP | 137.99 | 0.2 | 13.80 | g |
| Monobasic | | | | | |
| Deionized Water | H₂O | — | — | 500 | ml |

TABLE 6B
Calcium Chloride Stock Solution (200 mM)

| | | Molecular | Target | Target | |
|---|---|---|---|---|---|
| Raw Materials | Formula | Weight | Molarity | (500 ml) | |
| Calcium Chloride, Dihydrate | CaCl * | 147.02 | 0.2 | 14.70 | g |
| | 2H₂O | | | | |
| Deionized Water | H₂O | — | — | 500 | ml |

The remineralization solution functioned as an artificial saliva. Calcium and Phosphorus levels were theoretically equal 32 ppm Ca and 74 ppm P.

TABLE 7
Remineralization Solution

| | | Molecular | Target | Target | |
|---|---|---|---|---|---|
| Materials | Formula | Weight | Molarity | (1 Liter) | |
| Calcium Nitrate, | Ca(NO₃)₂ * | 236.15 | Ca 0.8 | 0.1889 | g |
| Tetrahydrate | 4H₂O | | | | |
| Potassium Phosphate | KH₂PO₄ | 136.09 | 2.4 | 0.3266 | g |
| Potassium Chloride | KCl | 74.55 | 130 | 9.69 | g |
| BisTris (CAS 6976-37-0) | C₈H₁₉NO₅ | 209.24 | 20 | 4.18 | g |
| Hydrochloric Acid, | HCl | 36.46 | — | 0.40 | ml |
| Concentrated | | | | | |
| Deionized Water | H₂O | — | — | 1000 | ml |

### Specimen Preparation

Ground and polished human enamel cores (3-4 mm round) mounted in acrylic rods were used for this procedure. Rods were inserted with the specimen end down and the non-specimen end pushed up through the underside of the plate lid. Care was taken to avoid touching the specimen end of the rod during this procedure. Specimens were positioned in such a way so that when the lid is placed on the reservoir, the end of the specimen was not touching the bottom of the reservoir and is approximately 5 mm above the bottom surface. Placement was important because if the specimen is placed much higher it will not adequately reach the solution during treatment. To store, loaded lids were placed on top of single-well reservoirs with a small amount of deionized water to maintain a humid environment. The reservoirs with lids were placed in the refrigerator for storage.

### F Presoak

10 mL per specimen of fluoride presoak solution was added to a deep-well reservoir (100 mL if soaking 10 specimens). The lid containing specimens was placed on top of the deep-well reservoir making sure the end of each specimen was submerged in the solution. The specimens were incubated at 37° C. with gentle shaking for 18-24 hours. After incubation, the specimens were removed from the fluoride presoak solution and washed briefly in a separate reservoir containing deionized water. The specimen lids were stored as before in a humid environment in the fridge until cycling began.

### Reagent Preparation, Day 1

Each treatment group had a designated and labeled treatment reservoir, wash reservoir, demin 24-deep-well plate and remin reservoir. Wash reservoirs were filled with ˜80 mL of deionized water. Remin reservoirs were filled with 10 mL per specimen (i.e. 100 mL for 10 specimens in a group) of remineralization solution. Before filling demin plates, the demineralization solution was checked on a calibrated pH meter to insure it was pH 4.30 (+/−0.01). The pH of this solution was readjusted, if necessary, prior to use. 5 mL of pH adjusted demineralization solution was added to each well of the plate in which a specimen was placed. All containers were covered with lids to avoid evaporation until use.

### Day 1

There was no toothpaste treatment prior to demin cycle 1. To begin cycle 1, specimen lids were removed from storage, rinsed in deionized water, and then placed directly onto labeled and filled demin containers. The demin plates were incubated at 37° C. without agitation for 6 hours.

Slurry Making: Dentifrice slurries (25% paste in water) were prepared by mixing 1 part by weight dentifrice (15 g) with three parts by volume water (45 mL) into a 100 mL beaker with a cross shaped Teflon coated stir bar. The slurry was mixed on a non-aerating mixer for a minimum of 5 minutes, or until thoroughly mixed, at a speed fast enough to completely disperse the paste but without creating excessive foam. The total volume of the slurry was approximately 60 mL per treatment group).

Wash, PM Treatment, Wash: At the end of the 6 hr demin period, the specimen lid was from the demin container and placed onto the wash reservoir for that group containing deionized water. The specimens were washed by shaking on the titer plate shaker for approx. 20 seconds before treatment. The mixed slurry was poured into the treatment reservoir and the lid with specimens was placed on top while care was taken to making sure the enamel end is immersed in the slurry. The treatment plate was shaken on the titer plate shaker robustly for 1 minute. After the 1-minute treatment, the lid with specimens was removed from the slurry and placed back on the labeled wash reservoir for that group containing deionized water. The specimens were washed by shaking for 20 seconds. Each treatment group was washed in a different wash reservoir to avoid contamination between paste formulas.

Remineralization Period: After the treatment and wash, each lid with specimens on top were placed in the filled remin reservoirs containing remineralization solution and incubated for 18 hr at 37° C.

Aliquot Demin Solution: One ml of the used demin solution from each specimen well was aliquoted into a 15 mL tube for ICP analysis. Filled tubes were stored in the refrigerator until analysis.

### Day 2, 3, 4 and 5 Cycling

Day 2, 3 and 4 had AM and PM toothpaste treatments. Day 5 will only had an AM treatment. The cycling protocol below was used for each cycle.

Preparation: Remin reservoirs were filled with 10 mL per specimen (i.e. 100 mL for 10 specimens in a group) of remineralization solution. Demin reservoirs were filled with 5 mL of pH adjusted demineralization solution. Wash reservoirs were filled with 80 mL of deionized water. All containers were covered with lids to avoid evaporation until use.

Slurry Making: Dentifrice slurries (25% paste in water) were prepared by mixing 1 part by weight dentifrice (15 g) with three parts by volume water (45 mL) into a 100 mL beaker with a cross shaped Teflon coated stir bar. The slurry was mixed on a non-aerating mixer for a minimum of 5 minutes, or until thoroughly mixed, at a speed fast enough to completely disperse the paste but without creating excessive foam. The total volume of the slurry equaled approximately 60 mL per treatment group (this volume was the minimum necessary to fill the treatment reservoir to an appropriate level).

Wash and AM Treatment: Specimens were from overnight remin container (used remin solution was discarded) and the lid with specimen was placed on the labeled wash reservoir for that group containing deionized water and shaken on a titer plate shaker for ˜20 seconds before treatment. Slurries were poured into the labeled treatment reservoir and the lid with specimens was placed on top. Care was taken to ensure the enamel end was immersed in the slurry and the treatment slurries were shaken on the titer plate shaker at speed 3 for 1 minute. The slurries were made fresh just prior to each treatment throughout the cycling process.

Wash: After the 1-minute treatment, the lid with specimens was removed from the slurry and placed back on the labeled wash reservoir for that group containing deionzed water. Samples were washed by shaking on the titer plate shaker for approx. 20 seconds. Each treatment group was washed in a different wash reservoir to avoid contamination between paste formulas.

Demineralization Period: After washing, each lid with specimens was placed on top of the appropriate, labeled 24-deep well plate containing 5 mL demineralization solution per well and incubated at 37° C. without agitation for 6 hr.

Wash, PM Treatment, Wash: Near the end of the 6 hour demin period, fresh treatment slurries were be prepared as described herein. Wash containers were refilled with fresh MQ water. Wash, treat and wash the specimens again as described herein.

Remineralization Period: After the PM treatment and wash, each lid with specimen was placed on top of the appropriate, labeled and filled remin reservoir containing remineralization solution and incubated overnight (18 hrs) at 37° C.

Aliquot Demin Solution: One mL of the used demineralization solution from each specimen well was aliquoted into a 15 mL tube for ICP analysis

Repeat: The previous steps were repeated for days 3 and 4. On day 5, only the AM portion of the treatment cycle was repeated for a total of 10 cycles.

The used demineralization on cycles from each treatment cycles were analyzed by ICP-MS to determine the total calcium in each solution. The average blank demineralization solution value was subtracted from the total calcium in each solution. The calcium loss from each cycle was added together to get the total calcium loss throughout the cycling procedure.

The same procedure was used for dentin pH cycling, except dentin samples were utilized.

TABLE 8
Calcium Loss during pH Cycling

| | | Average Ca | | Average Ca | |
|---|---|---|---|---|---|
| | | Loss in | δ Ca Loss in | Loss in | δ Ca Loss |
| | Summary of | Enamel | Enamel | Dentin | in Dentin |
| Examples | Ingredients | (ppm-days) | (ppm-days) | (ppm-days) | (ppm-days) |
| Ex. 1 | SnF₂, SnCl₂, | 24.8 | 6.7 | 49.6 | 10.9 |
| | Oxalate | | | | |
| Ex. 2 | NaF, Oxalate | 25.9 | 7.9 | 53.7 | 12.7 |
| Ex. 3 | Oxalate | 54.6 | 7.2 | 68.2 | 7.7 |
| Ex. 4 | SnF₂, SnCl₂ | 19.1 | 3.5 | 56.4 | 9.8 |
| CCP diluted | 100 ppm NaF | 71.4 | 4.9 | 76.3 | 25.4 |
| CCP | 1100 ppm NaF | 43.1 | 6.2 | 65.6 | 13.2 |
| Colgate | 2800 ppm NaF | — | — | 48.2 | 9.1 |
| PreviDent | | | | | |
| Diluted | | | | | |
| Colgate | 5000 ppm NaF | — | — | 41.0 | 7.4 |
| PreviDent | | | | | |
| USP SnF2 | SnF₂ | — | — | 62.6 | 9.3 |

TABLE 8 shows the measured enamel and dentin calcium loss throughout the pH cycling protocol. Unexpectedly, the addition of dicarboxylic acid, such as oxalate, to fluoride containing oral care compositions led to less calcium loss, which suggests an enhanced anticaries benefit. For example, Ex. 4 (SnF₂/SnCl₂) had 56.4 ppm of dentin Ca loss while Ex. 1 (SnF₂/SnCl₂+Oxalate) had only 49.6 ppm of dentin cavity loss. The addition of oxalate to NaF was more dramatic with an improvement of dentin Ca loss of 65.6 ppm (CCP NaF 1100 ppm) to 53.7 ppm (Ex. 2, NaF 1100 ppm+Oxalate) and an improvement of enamel Ca loss of 43.1 ppm (CCP NaF 1100 ppm) to 25.9 ppm (Ex. 2, NaF 1100 ppm+Oxalate).

Additionally, dicarboxylic acid, such as oxalate, unexpectedly does provide a small anticaries benefit as demonstrated by a lower Ca loss from enamel/dentin. For example, Ex. 3 (oxalate only) had a lower amount of Ca loss from enamel, 54.6 ppm, than the diluted CCP sample (100 ppm F), 71.4 ppm, and a lower amount of Ca loss from dentin, 68.2 ppm, than the diluted CCP sample (100 ppm F), 76.3 ppm. While it is known that, that higher fluoride levels, such as 5000 ppm, can improve anticavity benefit, it is unexpected that dicarboxylic acid can provide an anticavity benefit on its own or improve the anticavity benefit of fluoride when used in combination.

Desirable compositions include oral care compositions that result in a Ca loss in dentin and/or enamel of less than about 50 ppm, less than about 45 ppm, less than about 40 ppm, less than about 30 ppm, or less than about 25 ppm as determined by the pH cycling method described herein.

### Enamel Softening

The enamel softening method is used to determine the potential of oral care compositions to damage (or not to damage) dental enamel with repeated exposure. A microhardness tester was used to determine the change in hardness of dental enamel following cyclic exposure to the oral care compositions in TABLE 1A and TABLE 1B, in comparison to the control compositions: 1) deionized water; and 2) 1% citric acid solution.

A core of sound human enamel with a diameter of 3-4 mm was extracted from whole human teeth. The cores were mounted in dental acrylic and the surfaces were ground using 600 grit paper. Increasingly fine lapping papers were then used to polish the surface to a 1 μm polish. Samples were sonicated in deionized water for 30 min. Enamel specimens were then rinsed with deionized water and wiped to remove any residual polish. Each enamel specimen was inspected and samples with large cracks or uneven calcification were discarded. Enough specimens were prepared to provide 8 specimens for each treatment group. Enamel specimens were stored in an airtight container above a small amount of deionized water (˜1-5 mL) in a standard laboratory refrigerator (˜2-4° C.).

The artificial saliva solution of TABLE 9 was prepared on the day before the experiment. Also on the day before the experiment, the Vickers hardness of each enamel specimen was measured using a hardness indenter at three separate locations spread across the enamel surface. A 50 g load was applied for 10 seconds, and the diagonal lengths of the resulting indents were measured using a 20× magnification objective. The average Vickers hardness of the three indents was used to determine the average pre-cycling enamel hardness. Enamel specimens were then assigned to treatment groups such that the average hardness of each treatment group and the standard deviation of the average hardness were similar.

TABLE 9
Artificial Saliva Solution

| | | Molecular | Target | Target | |
|---|---|---|---|---|---|
| Materials | Formula | Weight | Molarity | (1 Liter) | |
| Calcium Nitrate, | Ca(NO₃)₂ * | 236.15 | Ca 0.8 | 0.3540 | g |
| Tetrahydrate | 4H₂O | | | | |
| Potassium Phosphate | KH₂PO₄ | 136.09 | 2.4 | 0.1230 | g |
| Potassium Chloride | KCl | 74.55 | 130 | 11.18 | g |
| BisTris | C₈H₁₉NO₅ | 209.24 | 20 | 4.185 | g |
| (CAS 6976-37-0) | | | | | |
| Hydrochloric Acid, | HCl | 36.46 | — | Adjust to | |
| Concentrated | | | | pH 7 | |
| Deionized Water | H₂O | — | — | 1000 | ml |

On the day of the cycling treatments, each treatment group was removed from the storage container and rinsed. The samples were cycled for a total of six rounds through the following procedure:
1) Specimens were treated by group in a 1:3 well-mixed slurry of toothpaste to water under quiescent conditions. The control group specimens were treated with deionized water or 1% citric acid solution.
2) The specimens were rinsed with copious amounts of water until residual toothpaste was removed.
3) The specimens were treated in quiescent saliva for 55 minutes.
4) The specimens were rinsed with copious amounts of water until residual saliva was removed.
   Following the sixth round of this exposure protocol, the specimens were stored in an airtight container over, but not touching, a small amount of deionized water.

On the day following the cycling experiment, the post-cycling hardness was obtained for each specimen using a similar procedure to that described for the pre-cycling hardness measurements. The change in hardness was calculated for each specimen by subtracting the pre-cycling hardness from the post-cycling hardness measurement. The average change in specimen hardness with respect to treatment and its standard deviation were then determined.

The statistical grouping was then determined using JMP with an α=0.05 in a student's t-test. The cycling was repeated if the average change in specimen hardness for the 1% citric acid positive control was not significantly different from the deionized water negative control. Statistical significance was checked for the difference between the dentifrice-slurry-treated specimens and those in the negative control, deionized water, treatment group. Those treatments that were significantly different than the negative control were determined to detrimentally soften the enamel surface.

The results of the enamel softening experiment are given in TABLE 10. At pH ca. 4.5 an oxalate version of a low pH toothpaste was found to damage enamel relative to the water negative control. Because of these data, we find it necessary to limit the pH range of oxalate-containing toothpastes to prevent softening of the enamel surface.

TABLE 10
Enamel Softening Results Showing Change in Surface
Microhardness (ΔSMH).

| | Slurry | | Statistical | |
|---|---|---|---|---|
| Treatments | pH | Δ SMH | Grouping | Study |
| Water | 5.3 | −15.02 | C | 1 |
| 1% Citric Acid | 2.19 | 175.68 | A | 1 |
| Ex. 5 | 4.56 | 42.42 | B | 1 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
(a) dicarboxylic acid; and
(b) fluoride,
wherein the pH of the oral care composition is from about 4 to about 7.

2. The oral care composition of claim 1, wherein the dicarboxylic acid comprises a compound with the formula HO₂C—R—CO₂H.

3. The oral care composition of claim 2, wherein R is aliphatic, aromatic, or combinations thereof.

4. The oral care composition of claim 1, wherein the dicarboxylic acid comprises oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, fumaric acid, terephthalic acid, salts thereof, or combinations thereof.

5. The oral care composition of claim 1, wherein the oral care composition comprises a dentifrice composition, a unit-dose oral care composition, an emulsion composition, a leave-on oral care composition, or combinations thereof.

6. The oral care composition of claim 1, wherein the pH is from about 4.5 to about 6.

7. The oral care composition of claim 1, wherein the pH is from about 4.5 to about 5.5

8. The oral care composition of claim 1, wherein the fluoride comprises stannous fluoride, sodium fluoride, sodium monofluorophosphate, amine fluoride, or combinations thereof.

9. The oral care composition of claim 1, wherein the oral care composition comprises tin.

10. The oral care composition of claim 9, wherein the tin comprises stannous fluoride, stannous chloride, or combinations thereof.

11. The oral care composition of claim 1, wherein the oral care composition comprises polyphosphate.

12. The oral care composition of claim 11, wherein the polyphosphate comprises pyrophosphate, tripolyphosphate, tetrapolyphosphate, hexametaphosphate, or combinations thereof.

13. The oral care composition of claim 1, wherein the oral care composition is free of, essentially free of, or substantially free of polyphosphate.

14. The oral care composition of claim 1, wherein the oral care composition comprises zinc.

15. The oral care composition of claim 14, wherein the zinc comprises zinc citrate, zinc lactate, zinc oxide, zinc phosphate, or combinations thereof.

16. The oral care composition of claim 1, wherein the oral care composition is free of, essentially free of, or substantially free of zinc.

17. The oral care composition of claim 1, wherein the oral care composition comprises monodentate ligand, polydentate ligand, or combinations thereof.

18. The oral care composition of claim 17, wherein the oral care composition has a tin to monodentate ligand to polydentate molar ratio of from about 1:0.5:0.5 to about 1:5:5.

19. The oral care composition of claim 1, wherein the oral care composition comprises thickening agent.

20. The oral care composition of claim 19, wherein the thickening agent comprises polysaccharide, polymer, silica thickener, or combinations thereof.

21. The oral care composition of claim 1, wherein the oral care composition comprises abrasive.

22. The oral care composition of claim 21, wherein the abrasive comprises silica abrasive, calcium abrasive, or combinations thereof.

23. The oral care composition of claim 22, wherein the silica abrasive comprises precipitated silica.

24. The oral care composition of claim 22, wherein the calcium abrasive comprises calcium carbonate, calcium pyrophosphate, calcium phosphate, hydroxyapatite, or combinations thereof.

25. The oral care composition of claim 1, wherein the oral care composition comprises amino acid.

26. The oral care composition of claim 25, wherein the amino acid comprises basic amino acid, acidic amino acid, neutral amino acid, or combinations thereof.

27. The oral care compositions of claim 26, wherein the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

28. The oral care composition of claim 1, wherein the oral care composition comprises whitening agent.

29. The oral care composition of claim 28, wherein the whitening agent comprises peroxide, polyphosphate, or combinations thereof.

30. The oral care composition of claim 1, wherein the oral care composition comprises humectant.

31. The oral care composition of claim 30, wherein the humectant comprises glycerin, sorbitol, erythritol, xylitol, butylene glycol, propylene glycol, polyethylene glycol, or combinations thereof.

32. The oral care composition of claim 1, wherein the oral care composition comprises no added water.

33. The oral care composition of claim 1, wherein the oral care composition comprises water.

34. The oral care composition of claim 1, wherein the oral care composition comprises up to 45%, by weight of the composition, of water.

35. The oral care composition of claim 1, wherein the oral care composition provides less than about 50 ppm, less than about 45 ppm, less than about 40 ppm, less than about 30 ppm, or less than about 25 ppm of calcium loss.

36. An oral care kit comprising:
(a) a first oral care composition comprising fluoride; and
(b) a second oral care composition comprising dicarboxylic acid.

37. The oral care kit of claim 36, wherein the oral care kit comprises instructions directing a user to apply the first oral care composition to the oral cavity followed by applying the second oral care composition to the oral cavity in a single oral care session.

38. The oral care kit of claim 37, wherein the single oral care session is from about 1 minute to about 3 minutes.

39. The oral care kit of claim 38, wherein the first oral care composition comprises tin, abrasive, humectant, water, whitening agent, amino acid, polyphosphate, zinc, thickening agent, or combinations thereof.

40. The oral care kit of claim 39, wherein the second oral care composition is free of, essentially free of, or substantially free of fluoride.

41. The oral care kit of claim 36, wherein the dicarboxylic acid comprises a compound with the formula HO₂C—R—CO₂H.

42. The oral care composition of claim 41, wherein R is aliphatic, aromatic, or combinations thereof.

43. The oral care composition of claim 36, wherein the dicarboxylic acid comprises oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, fumaric acid, terephthalic acid, glutaric acid, salts thereof, or combinations thereof.

44. The oral care kit of claim 36, wherein the oral care kit comprises instructions directing a user to apply the second oral care composition to the oral cavity followed by applying the first oral care composition to the oral cavity in a single oral care session.
